(19) United States
(12) Patent Application Publication
(10) Pub. No.: US 2025/0092313 A1
(43) Pub. Date: Mar. 20, 2025

(54) MIXTURES OF CLEAVABLE QUATERNARY AMMONIUM COMPOUNDS USEFUL AS SURFACTANTS

(71) Applicant: SPECIALTY OPERATIONS FRANCE, Lyon (FR)

(72) Inventors: Olivier Back, Lyon (FR); Christopher Boardman, Bebington (GB)

(73) Assignee: SPECIALTY OPERATIONS FRANCE, Lyon (FR)

(21) Appl. No.: 18/580,893

(22) PCT Filed: Jul. 13, 2022
(86) PCT No.: PCT/EP2022/069643

§ 371 Date: Jan. 19, 2024

(30) Foreign Application Priority Data
Jul. 20, 2021 (EP) 21186561.3

(51) Int. Cl. C09K 23/18; C07C 227/18

(52) U.S. Cl. CPC C09K 23/18; C07C 227/18

(57) ABSTRACT

The invention concerns new mixtures of quaternary ammonium compounds with surfactant properties and improved biodegradability.

The present invention relates to mixtures of ammonium compounds, in particular quaternary ammonium compounds derivable from internal ketones, themselves obtainable from mixtures of fatty acids or their derivatives, processes to produce such mixtures and the use of these mixtures as surfactants, alone or in admixture with other surfactants.

Ammonium compounds which have surfactant properties and can be used in respective applications have been described in the literature and are available commercially in a variety of different types from various suppliers.

JP3563473 B2 discloses a quaternary ammonium salt represented by the formula R₁R₂R₃N⁺—(CH₂)ₙ—COO-(AO)ₘ—CHR₄R₅ in which R₁, R₂ and R₃ are each an alkyl group or hydroxyalkyl group having 1-4 carbon atoms, R₄ and R₅ are each a straight or branched alkyl group or alkenyl group having 7 to 35 carbon atoms, A is a straight or branched alkanediyl group having 2-3 carbon atoms, X is an anionic group, n is an integer between 1 and 6, and m is a number between 0 and 20 that indicates the average number of moles of alkylene oxide. R₄ and R₅ can be pentadecyl, heptadecyl or mixtures thereof. This surfactant is said to be usable to impart fibers with softness while having a good biodegradability.

Alkoxylated quaternary ammonium salts presented in said japanese document are low performance products with hydrolytic stability issues and furthermore the production of such products induces formation of by-products like dioxane which is toxic, suspected to be carcinogenic, persistent and is therefor under strong regulatory pressure. With regards to the above quaternary ammonium compounds with m=0 as surfactants, the Applicant discovered that it is difficult to find a good combination of surfactant properties on one hand and biodegradability on the other hand. Biodegradability has become more and more important in the recent past due to the desire of customers to have more environmentally friendly products. The improvement in biodegradability should not negatively affect the surfactant properties.

It was thus an object of the present invention to provide a new solution with good surfactant properties and an excellent biodegradability.

This object is achieved with a specific mixture of compounds of formula I defined below.

### BRIEF DESCRIPTION

A first object of the present invention is a mixture of compounds in accordance with the present invention having the formula I

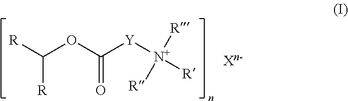

wherein R groups, which may be the same or different at each occurrence, are C₁₅ or C₁₇ aliphatic group,
Y is a divalent C₁-C₆ aliphatic group,
R′, R″ and R′″, which may be the same or different, are hydrogen or a C₁ to C₄ alkyl group,
Xⁿ⁻ is a counter-anion selected from the group consisting of
   a halide (n=1),
   a hydrocarbylsulfate anion of formula R^{a}—O—SO₂—O⁻ wherein R^{a} denotes a C₁-C₂₀, preferably C₁-C₆, hydrocarbyl group which can be optionally halogenated (n=1),
   a hydrocarbylsulfonate anion of formula R^{a}—SO₂—O^{—} wherein R^{a} denotes a C₁-C₂₀, preferably C₁-C₆, hydrocarbyl group which can be optionally halogenated (n=1),
   a sulfate anion of formula SO₄²⁻ (n=2)
   a hydrogensulfate (or bisulfate) anion of formula HSO₄⁻ (n=1),
   a carbonate anion of formula CO₃²⁻ (n=2)
   a hydrogencarbonate (or bicarbonate) anion of formula HCO₃⁻ (n=1)
   a dihydrogenphosphate anion of formula H₂PO₄⁻ (n=1)
   a hydrogenphosphate anion of formula HPO₄²⁻ (n=2)
   a phosphate anion of formula PO₄³⁻ (n=3)
   an organic carboxylate anion of formula R^{a}(CO₂⁻)ₙ wherein R^{a} denotes a C₁-C₂₀, preferably C₁-C₆, hydrocarbyl group which can be optionally substituted by an heteroatom containing group (n=1, 2 or 3),
   and mixtures thereof,
n is an integer which is equal to 1, 2 or 3, depending on the nature of the counter-anion and
said mixture comprising from 20 to 95% mol of compounds of formula I wherein both R are both C₁₅ aliphatic groups.

Another object of the present invention is a process to produce the above mixture of compounds of formula I, wherein said process is starting from a mixture of fatty acids R—COOH, wherein R is a C₁₅ or C₁₇ aliphatic group and said mixture of fatty acids comprising from 45 to 98% mol of R—COOH wherein R is a C₁₅ aliphatic group.

The present invention also concerns the use of the above mixture of compounds of formula (I) as surfactant.

All preferred embodiments of the present invention are also detailed hereinafter and apply to all categories of claims.

### DETAILED DESCRIPTION

The aliphatic groups R may be free of any double bond and of any triple bond. Alternatively, the aliphatic groups R may comprise at least one —C═C— double bond and/or at least one —C═C— triple bond.

The aliphatic groups R are advantageously chosen from alkyl groups, alkenyl groups, alkanedienyl groups, alkanetrienyl groups and alkynyl groups.

The aliphatic groups R may be linear or branched, preferably linear.

Preferably, the aliphatic groups R are independently chosen from alkyl and alkenyl groups.

More preferably, the aliphatic groups R are independently chosen from linear alkyl and alkenyl groups.

Unsaturations on R group (R=alkenyl group) are rather favorable to biodegradability.

Acyclic aliphatic groups, more preferably linear aliphatic groups, still more preferably linear alkyl groups may be mentioned as preferred examples of substituents R. Excellent results were obtained when R were linear alkyl groups.

R′ is preferably H or a C₁ to C₄ alkyl group, preferably methyl or ethyl, more preferably methyl. Likewise, R″ is preferably H or a C₁ to C₄ alkyl group, preferably methyl or ethyl, more preferably methyl. Still likewise, R″ is preferably H or a C₁ to C₄ alkyl group, preferably methyl or ethyl, more preferably methyl. Preferably at least one, more preferably at least two, more preferably all three of R′, R″ and R″ are H or a C₁ to C₄ alkyl group, preferably methyl or ethyl, most preferably methyl.

Y is preferably an acyclic divalent C₁-C₆ aliphatic group, more preferably a saturated acyclic divalent C₁-C₆ aliphatic group, still more preferably a linear alkanediyl (commonly referred to as “alkylene”) C₁-C₆ group. Besides, Y has preferably from 1 to 4 carbon atoms. Exemplary Y are: methanediyl (commonly referred to as “methylene”), ethane-1,2-diyl (commonly referred to as “ethylene”) and ethane-1,1-diyl. Excellent results were obtained when Y was a methylene group.

Suitable Xⁿ⁻ are halides such as chloride, fluoride, bromide or iodide, methyl sulfate or methosulfate anion (CH₃—OSO₃⁻), methanesulfonate anion (CH₃—SO₃⁻), sulfate anion, hydrogensulfate anion (HSO₄⁻), carbonate anion, bicarbonate anion (HCO₃⁻), dihydrogenphosphate anion (H₂PO₄²⁻), hydrogenphosphate anion (HPO₄²⁻), phosphate anion or an organic carboxylate anion such as acetate, propionate, benzoate, tartrate, citrate, lactate, maleate or succinate.

The counter-anion Xⁿ⁻, when inorganic in its nature, like halide, sulfate anion, carbonate anion, bicarbonate anion (HCO₃⁻), hydrogensulfate anion, dihydrogenphosphate anion, hydrogenphosphate anion or phosphate anion, does not change biodegradability behavior of the corresponding quaternary ammonium compound.

When the counter-anion Xⁿ⁻ is organic, like methyl sulfate or methosulfate anion (CH₃—OSO₃⁻), methanesulfonate anion (CH₃—SO₃⁻), or a “short chain” organic carboxylate anion (R^{a}(CO₂⁻)ₙ) such as acetate (R^{a}═CH₃—, n=1), propionate (R¹═CH₃—CH₂—, n=1), tartrate (R^{a}═—CH(OH)—CH(OH)—, n=2), citrate (R^{a}═—CH₂—C(OH)(−)-CH₂—, n=3), lactate (R^{a}═CH₃—CH(OH)—, n=1), maleate (R^{a}═—CH═CH—, n=2) or succinate (R^{a}═—CH₂—CH₂—, n=2), the biodegradability is not expected to be significantly affected as the hydrogen and carbon content in the anion represents a weak proportion of the total hydrogen and carbon content of the whole salt (especially for polyanions). For organic carboxylate anions of formula R^{a}(CO₂⁻)ₙ, Rₐ is preferably C₁-C₆, more preferably C₁-C₄. Also, R^{a} is preferably a linear chain, and can be unsaturated because it is favourable for biodegradability.

As preferable list of counter-anion Xⁿ⁻ we can cite halides such as chloride (Cl⁻), fluoride (F⁻), bromide (Br⁻) or iodide (I⁻), methyl sulfate or methosulfate anion (CH₃—OSO₃⁻), methanesulfonate anion (CH₃—SO₃⁻), sulfate anion (SO₄²⁻), hydrogensulfate anion (HSO₄⁻), carbonate anion (CO₃²⁻), bicarbonate anion (HCO₃⁻), dihydrogenphosphate anion (H₂PO₄²⁻), hydrogenphosphate anion (HPO₄²⁻), phosphate anion (PO₄³⁻) or acetate (CH₃—COO^{—}).

According to one preferred embodiment, Xⁿ⁻ is a halide, preferably chloride, with n=1.

In the mixture according to the invention, it is advantageous that R groups are C₁₅ or C₁₇ alkyl groups and that the mixture comprises from 20 to 95% mol of compounds of formula I wherein both R groups are C₁₅ alkyl groups.

The best results are obtained when the mixture according to the invention is such as R groups are C₁₅ or C₁₇ linear alkyl groups and said mixture comprises from 20 to 95% mol of compounds of formula I wherein both R groups are C₁₅ linear alkyl groups.

As shown in the experimental part below, if the mixture is containing less than 20% mol of compounds of formula I wherein both R groups are C₁₅ linear alkyl groups, the biodegradability performance is not reached. Experiments have also demonstrated that above the limit of 95%, the hydrophobicity of the mixture is impacted, which will reduce the performance as surfactant in certain applications. The optimal balance is not easy to reach, as we both need biodegradability and surfactant performances. Indeed, if the mixture is not containing a minimum of compounds of formula I wherein R are C₁₇ linear alkyl groups, the CMC (Critical Micelle Concentration) is high and we would need to introduce a higher quantity of surfactant in the targeted formulation to reach the performance in the application.

In the mixture according to the invention, excellent results are obtained when the mixture comprises from 20 to 60% mol, preferably 30 to 50% mol of compounds of formula I wherein both R groups are C₁₅ aliphatic groups, preferably alkyl groups and notably linear alkyl groups.

According a preferred embodiment, the mixture according to the invention comprises:
from 20 to 95% mol of compounds of formula I wherein both R groups are C₁₅ linear alkyl groups, preferably from 20 to 60% mol, more preferably from 30 to 50% mol,
from 4.9 to 50% mol of compounds of formula I wherein one R group is a C₁₅ linear alkyl group and the other R group is a C₁₇ linear alkyl group, preferably from 35 to 50% mol, more preferably from 41 to 50% mol and
from 0.1 to 31% mol of compounds of formula I wherein both R groups are C₁₇ linear alkyl groups, preferably from 5 to 31% mol, more preferably from 9 to 20% mol.

The mixture according to the invention can further comprise less than 5% mol of compounds of formula I wherein at least one of the R groups, which may be the same or different at each occurrence, are C₇ to C₁₃ aliphatic groups, preferably less than 2% mol. Those products are by-products that come from the raw materials used. Indeed, when the fatty acid cut used as starting material contains low quantities of one or more fatty acid(s) based on C₇ to C₁₃ aliphatic groups, all the possible internal ketones that can be obtained by the coupling of any of this one or more fatty acid(s) based on C₇ to C₁₃ aliphatic group with any fatty acid contained in the cut are produced during the step of decarboxylative ketonization.

The mixture according to the invention can further comprise less than 5% mol of compounds of formula I wherein at least one of the R groups, which may be the same or different at each occurrence, are C₁₉ to C₂₁ aliphatic groups, preferably less than 2% mol. Those products are by-products that come from the raw materials used. As previous explained, when the fatty acid cut used as starting material contains low quantities of one or more fatty acid(s) based on C₁₉ to C₂₁ aliphatic groups, all the possible internal ketones that can be obtained by the coupling of any of this one or more fatty acid(s) based on C₁₉ to C₂₁ aliphatic groups with any fatty acid contained in the cut are produced during the step of decarboxylative ketonization (see step a. below in the description).

According to a particular embodiment of the invention, the mixture of compounds of formula I does essentially contain compounds of formula I wherein R groups, which may be the same or different at each occurrence, are C₁₅ or C₁₇ linear alkyl groups. It means that other compounds are representing less than 2% mol, preferably less than 1% mol.

The above defined mixture according to the invention is displaying good surfactant properties on one side and good biodegradability on the other side.

In the experimental part, it is shown that by a careful control of the hydrocarbon average chain length (R—CH—R) (notably through the careful choice of the starting fatty acid) a good balance between surfactant property on one side and biodegradability on the other side can be achieved. For example, starting from a C₁₆:C₁₈ fatty acid mixture, a minimal amount of C₁₆ is necessary in the starting fatty acid to achieve readily biodegradation for the final compound. At the same time, a minimum amount of C₁₈ is also necessary in the starting fatty acid to achieve surfactant properties for the final compound.

In addition, it is important to note that, at least for industrial usage, the starting raw material of the mixture of the present invention is coming from renewable resources, typically palm oil cut of fatty acids, containing both C₁₆ and C₁₈ fatty acids. C₁₆ and C₁₈ fatty acids are very difficult to isolate from one another: it is highly energy consuming and expensive, resulting in a non-sense on an industrial point of view.

The mixture of compounds of formula I in accordance with the present invention can be obtained by a variety of processes. Preferred processes for the manufacture of the compounds of the present invention include the reaction of an internal ketone of formula VI: R—C(═O)—R (VI), which internal ketone may preferably be obtained by decarboxylative ketonization of a mixture of fatty acids, fatty acid derivatives or a mixture thereof. A suitable process for the manufacture of internal ketones following this route is diclosed in US 2018/0093936 to which reference is made for further details. Anyway, such a mixture of compounds of formula I as defined above is advantageously obtained through a process starting from a mixture of fatty acids R—COOH, wherein R is a C₁₅ or C₁₇ aliphatic group and said mixture of fatty acids comprising from 45 to 98% mol of R—COOH wherein R is a C₁₅ aliphatic group.

It is particularly preferred that said process is starting from a mixture of fatty acids R—COOH, wherein R is a C₁₅ or C₁₇ linear alkyl group and said mixture of fatty acids comprising from 45 to 78% mol, more preferably from 55 to 71% mol of R—COOH wherein R is a C₁₅ linear alkyl group.

The process of the present invention can be a process including: 1) Piria ketonization (or decarboxylative ketonization) of a mixture of fatty acids described above, 2) Ketone hydrogenation to a mixture of secondary fatty alcohols, 3) Alcohol esterification, notably with chloroacetic acid (in the case Y is methylene), 4) Condensation of the mixture of monoesters, notably chloroesters with an amine, 5) Optionally anion exchange to afford the desired quaternary ammonium mixture of compounds of formula I.

The process starts with a Piria ketonization followed by hydrogenation, and esterification to obtain a mixture of monoesters. The esterification reaction step is followed by an amine condensation step to convert the monoester into a mixture of compounds that can comply with formula I or that can be further reacted through an anion exchange reaction to comply with formula I. This is a multi-step process plugged on Piria technology. It has the advantage of being salt-free when no step of anion exchange is performed and relying on chemical transformations which can be easily performed.

The global process according to the invention can comprise the following steps:
a. decarboxylative ketonization of a mixture of fatty acids R—COOH, wherein R is a C₁₅ or C₁₇ aliphatic group and wherein said mixture of fatty acids comprises from 45 to 98% mol of R—COOH with R being a C₁₅ aliphatic group, in presence of a metal catalyst thus obtaining a mixture of internal ketones of formula VI: R—C(═O)—R (VI), wherein R groups which may be the same or different at each occrence are as defined above,
b. Hydrogenation of the mixture of internal ketones of formula VI obtained at step a. in presence of H₂ and a catalyst thus obtaining a mixture of secondary alcohols of formula V: R—CH(OH)—R (V), wherein R groups which may be the same or different at each occurence are as defined above,
C. Esterification of the mixture of secondary alcohols of formula V obtained at step b. with a carboxylic acid reagent of formula IV:

[L-Y—CO₂H]^{(t-1)-}[U^{u+}]_{(t-1)/u} (IV)
wherein L is a leaving group,
t is an integer which is equal to 1 or which is equal or superior to 2,
U^{u+} is a cation,
u is an integer fixing the positive charge of the cation,
Y is as defined in claim 1 or 4 and
R groups are as previously described,
   thus obtaining a mixture of monoesters of formula III:

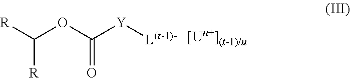

wherein R,Y, L, t, U, and u are as previously described,
d. Condensation of the mixture of monoesters of formula III obtained at step c. with an amine of formula R′R″R′″N, wherein R′, R″ and R′″ which may be the same or different, are hydrogen or a C₁ to C₄ alkyl group to obtain a mixture of compounds of formula II:

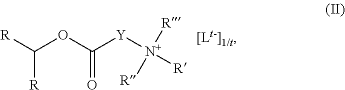

wherein R, R′, R″, R′″, Y, L and t are as previously described,
e. Optionally a step of anion exchange by contacting the mixture of compounds of formula II obtained at step d. with a salt of formula [U′^{u′+}]_{n/u′}Xⁿ⁻ in order to substitute L^{t-} by Xⁿ⁻ when L^{t-} is different from Xⁿ⁻, X and n being as defined in any one of the preceding claims and U′^{u′+} is a cation, u′ is an integer fixing the positive charge of the cation, and
f. Recovering the mixture of compounds of formula I as defined above.

Further details on the process are given below.

### Process for Synthesis of the Mixture of Compounds of Formula I

a. Piria Ketonization

The basic reaction in the first step is:

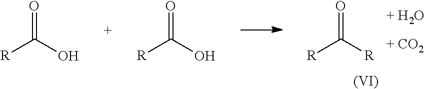

R groups have the same meaning as defined above.

This reaction has been thoroughly described in U.S. Pat. No. 10,035,746, WO 2018/087179 and WO 2018/033607 to which reference is made for further details.

b. Hydrogenation

The internal ketone mixture of formula VI is then subjected to hydrogenation which can be carried out under standard conditions known to the skilled person for hydrogenation reactions:

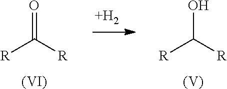

The hydrogenation reaction is conducted by contacting the internal ketone mixture of formula VI with hydrogen in an autoclave reactor at a temperature ranging from 15° C. to 300° C. and at a hydrogen pressure ranging from 1 bar to 100 bars. The reaction can be conducted in the presence of an optional solvent but the use of such solvent is not mandatory and the reaction can also be conducted without any added solvent. As examples of suitable solvents one can mention: methanol, ethanol, isopropanol, butanol, THF, methyl-THF, hydrocarbons, water or mixtures thereof. A suitable catalyst based on a transition metal should be employed for this reaction. As examples of suitable catalysts, one can mention heterogeneous transition metal based catalysts such as for example supported dispersed transition metal based catalysts or homogeneous organometallic complexes of transition metals. Examples of suitable transition metals are: Ni, Cu, Co, Fe, Pd, Rh, Ru, Pt, Ir. As examples of suitable catalysts one can mention Pd/C, Ru/C, Pd/Al₂O₃, Pt/C, Pt/Al₂O₃, Raney Nickel, Raney Cobalt etc. At the end of the reaction, the desired alcohol mixture of formula V can be recovered after appropriate work-up. The skilled person is aware of representative techniques so no further details need to be given here. Details of this process step can e.g. be found in U.S. Pat. No. 10,035,746 to which reference is made here.

The skilled person will select suitable reaction conditions based on his professional experience and taking into account the specific target compound to be synthesized. Accordingly, no further details need to be given here.

c. Esterification

The esterification of the above obtained alcohols mixture of formula V can thereafter be achieved by reacting said alcohols mixture of formula V with a carboxylic acid reagent of formula IV to obtain a mixture of monoester compounds of formula III:

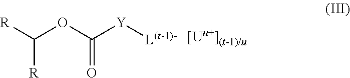

in accordance with the following scheme:

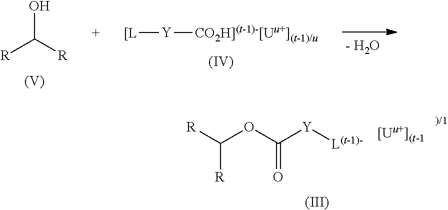

wherein, wherever present in the above compounds,
L is a leaving group,
t is an integer which is equal to 1 or which is equal or superior to 2,
U^{u+} is a cation,
u is an integer fixing the positive charge of the cation, and
R and Y are as previously described.

The esterification is performed by contacting the alcohol mixture of formula V with a carboxylic acid reagent of formula IV:

[L-Y—CO₂H]^{(t-1)-}[U^{u+}]_{(t-1)/u} (IV)
wherein L, Y, t, U^{u+} and u are as previously described.

When t is equal to 1, no cation is present. Otherwise said, the esterification reaction is performed by contacting the alcohol with a carboxylic acid of formula:

L-Y—CO₂H

In the case the leaving group L already carries a negative charge in the carboxylic acid reagent (this is the case when (t−1) is equal or superior to 1, i.e. when t is equal or superior to 2), a cation noted U^{u+} (with u preferably being 1, 2 or 3, more preferably 1) must be present in the reactant to ensure the electroneutrality. This cation may e.g. be selected from H⁺, alkaline metal cations (e.g. Na⁺ or K⁺), alkaline earth metal cations (e.g. Ca²⁺), Al³⁺ and ammonium, to mention only a few examples.

The nature of the leaving group L is not particularly limited provided next reaction step (i.e. amine condensation, as will be detailed later on) can occur. The leaving group L is advantageously a nucleofuge group. It can be notably chosen from
a halogen,
a (hydrocarbyloxysulfonyl)oxy group of formula R^{a}—O—SO₂—O— wherein R^{a} denotes a C₁-C₂₀ hydrocarbyl group which can be optionally halogenated,
a (hydrocarbylsulfonyl) oxy group of formula R^{a}—SO₂—O— wherein R^{a} denotes a C₁-C₂₀ hydrocarbyl group which can be optionally halogenated (such as in CF₃—SO₂—O—), and
an oxysulfonyloxy group of formula ⁻O—SO₂—O— (which is a leaving group L already carrying one negative charge on a terminal oxygen atom).

The hydrocarbyl group R^{a}, wherever present in here before formulae, can be notably an aliphatic group or an optionally substituted aromatic group such as phenyl or p-tolyl. The aliphatic group R^{a} is usually a C₁-C₆ alkyl group, which can be linear or ramified; it is often a linear C₁-C₄ alkyl, such as methyl, ethyl or n-propyl.

The leaving group L is preferably chosen from:
a halogen, such as fluorine, chlorine, bromine or iodine,
a (hydrocarbylsulfonyl) oxy group of formula R^{a}—SO₃— wherein R^{a} denotes a C₁-C₂₀ hydrocarbyl group, such as CH₃—SO₃— and
an oxysulfonyloxy group of formula ⁻O—SO₂—O—.

An example for a compound with t equal to 1 is CH₃—O—SO₃—CH₂—COOH which can be designated as 2-((methoxysulfonyl)oxy)acetic acid. As further examples of compounds in which t is equal to 1 and thus no cation is present, one can mention: chloroacetic acid, bromoacetic acid and 2-chloropropionic acid. Chloroacetic acid is the preferred reagent of formula IV.

An example for t being equal to 2 is sodium carboxymethylsulfate acid in which [L-Y—COOH]^{(t-1)-}[U^{u+}]_{(t-1)/u} is [O—SO₂—O—CH₂—COOH]⁻[Na⁺].

The reaction conducted during esterification step c. can be conducted in the presence of a solvent. However the presence of such solvent is not mandatory and the reaction can be also conducted without any added solvent. As example of suitable solvents one can mention: toluene, xylene, hydrocarbons, DMSO, Me-THF, THF or mixtures thereof.

The reaction is advantageously conducted under an inert atmosphere, such as a nitrogen or rare gas atmosphere. An argon atmosphere is an example of a suitable inert atmosphere.

The reaction can be conducted in the absence of any catalyst. A catalyst can also be employed during the reaction and suitable catalysts are Bronsted or Lewis acid catalysts. As preferred examples of catalysts one can mention: H₂SO₄, para-toluenesulfonic acid, methanesulfonic acid, trifluoromethanesulfonic acid, HCl, or heterogeneous acidic resins such as Amberlite® resins, AlCl₃, FeCl₃, SnCl₄, etc.

The total number of moles of the carboxylic acid reagent of formula IV which is contacted with the alcohol of formula V during the whole course of the reaction is advantageously no less than half of the total number of moles of alcohol; it is preferably at least as high as the total number of moles of alcohol, and it is more preferably at least twice higher than the total number of moles of alcohol. Besides, the total number of moles of carboxylic acid reagent which is contacted with the alcohol during the whole course of the reaction is advantageously at most ten times higher than the total number of moles of alcohol.

The reaction takes advantageously place in a reactor where the alcohol is in molten state. It has also been found advantageous that the reaction takes place in a reactor where the carboxylic acid reagent of formula IV is in molten state. Preferably, the reaction takes place in a reactor where both the alcohol and the carboxylic acid reagent are in molten state.

The esterification reaction can be conducted at a temperature ranging generally from about 20° C. to about 200° C. in the presence of an optional solvent. To allow for a sufficient reaction rate, the reaction is preferably conducted at a temperature which is of at least 60° C., more preferably at least 80° C., still more preferably at least 100° C. On the other hand, the Applicant has surprisingly found that conducting the reaction at a high temperature resulted in the formation of internal olefins as dehydration by-products and color build-up. Accordingly, the reaction is conducted at a temperature which is preferably below 180° C., more preferably below 160° C. and still more preferably of at most 150° C.

The whole reaction can be conducted at atmospheric pressure or at subatmospheric pressure in order to assist water removal and to drive the equilibrium toward completion. It is preferably conducted at atmospheric pressure or under vacuum, that is to say at a pressure from 10 kPa to the atmospheric pressure (about 1 atm=101.325 kPa). More preferably, it is conducted at atmospheric pressure.

At the end of the reaction, the desired mixture of monoester compounds of formula III, can be recovered after appropriate work-up and the skilled person is aware of representative techniques so that no further details need to be given here. For exemple, an appropriate work-up can consist on distilling the excess of carboxylic acid reagent under vacuum. Alternatively, the exces of carboxylic acid reagent can be removed by simple extraction of the crude organic mixture with an aqueous solution.

d. Amine Condensation

The mixture of monoester compounds of formula III can be converted into the mixture of compounds of formula II through the following reaction scheme:

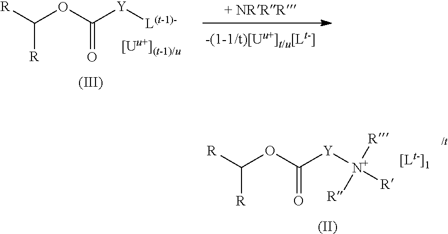

wherein R, R′, R″, R′″, Y, L, U, t and u are as described here before.

The amine condensation reaction is performed by contacting the mixture of intermediate monoester compounds of formula III with ammonia or an amine of formula NR′R″R″ where R′, R″ and R′″, which may be the same or different, are hydrogen or a C₁ to C₄ alkyl group, and preferred R′, R″ and R′″ are exactly as above defined in connection with the ammonium compound of formula I.

The reaction can be conducted at a temperature ranging from 15° C. to 250° C. in the presence of a suitable solvent. As example of a suitable solvent one can mention: THF, Me-THF, methanol, ethanol, isopropanol, butanol, ethyl acetate, DMSO, toluene, xylene or their mixture. Alternatively the reaction can be also conducted in the absence of any added solvent.

During this reaction, there is a nucleophilic attack of ammonia or of the amine that substitutes L^{(t-1)-} in the monoester; L^{(t-1)-} plays the role of the leaving group. L^{t-} becomes then the counter-anion of the final ammonium compound. In the case the leaving group already carries a negative charge in the monoester (this is the case when (t−1) is equal or superior to 1 or when t is equal or superior to 2) there is also formation of a salt as the by-product of the reaction with the general chemical formula [U^{u+}]_{t/u}[L^{t-}].

e. Optional Anion Exchange

In a preferred embodiment L^{t-} is equal to Xⁿ⁻ (in other words X is equal to L), which means that compounds of formula II are equal to compounds of formula I.

In this case the counterion Xⁿ⁻ of formula I is in fact coming from the leaving group L of previous steps. This is the case notably when Xⁿ⁻ is an halide, sulfate, hydrogensulfate, methanesulfonate, methosulfate, p-toluene sulfonate, dihydrogenphosphate, hydrogenphosphate, phosphate or organic carboxylate.

In another embodiment, the process of the invention comprises the step e. of anion exchange. For example when Xⁿ⁻ is a carbonate or bicarbonate, the mixture of compounds of formula I is obtained with an additional step e. of anion exchange in order to substitute L^{t-} by Xⁿ⁻.

For phosphate and carboxylate anions, both options are possible.

The anion exchange reaction during step e. can be conducted by contacting the mixture of compounds of formula II obtained at the end of step d. (which are basically compounds of formula I but containing the anion L^{t-} instead of Xⁿ⁻) to be substituted with a salt of formula [U′^{u′+}]_{n/u′}Xⁿ⁻ in an appropriate solvent system allowing one of the product of the anion exchange reaction to precipitate out (either the new compound of formula I with Xⁿ⁻ as the counter-anion or the salt by-product [U′^{u′+}]_{t/u′}L^{t-}) in order to drive the equilibrium toward completion. U′^{u′+} is a cation, u′ is an integer fixing the positive charge of the cation. This cation may e.g. be selected from H⁺, alkaline metal cations (e.g. Na⁺ or K⁺), alkaline earth metal cations (e.g. Ca²⁺), Al³⁺, Ag⁺ and ammonium, to mention only a few examples.

As example of solvents one can mention: water, methanol, ethanol, isopropanol, butanol, DMSO, acetone, aconitrile, ethyl acetate and their mixtures.

f. Recovering the Mixture of Compounds of Formula I

The final mixture of compounds of formula I can be recovered following an appropriate work-up known in the prior art.

A particularly preferred process according to the invention is a process comprising the following steps:
a. decarboxylative ketonization of a mixture of fatty acids R—COOH, wherein R is a C₁₅ or C₁₇ aliphatic group and wherein said mixture of fatty acids comprising from 45 to 98% mol of R—COOH with R being a C₁₅ aliphatic group, in presence of a metal catalyst thus obtaining a mixture of internal ketones of formula VI: R—C(═O)—R (VI), wherein R groups which may be the same or different at each occrence are as defined above,
b. Hydrogenation of the mixture of internal ketones of formula VI obtained at step a. in presence of H₂ and a catalyst thus obtaining a mixture of secondary alcohols of formula V: R—CH(OH)—R (V), wherein R groups which may be the same or different at each occurence are as defined above,
c. Esterification of the mixture of secondary alcohols of formula (V) obtained at step b. with a carboxylic acid reagent of formula (IV) being chloroacetic acid thus obtaining a mixture of monoesters of formula (III′)

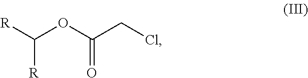

wherein R groups are as previously described,
d. Condensation of the mixture of monoesters of formula (III') obtained at step c. with an amine of formula R′R″R′″N, wherein R′, R″ and R′″ which may be the same or different, are hydrogen or a C₁ to C₄ alkyl group to obtain directly a mixture of compounds of formula (I′):

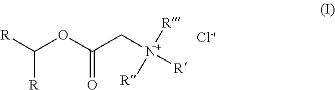

wherein R groups are as previously described.

This preferred process is salt free and chemical transformations can be easily performed.

### Other Processes for the Preparation of the Mixture of Compounds of Formula I According to the Invention

An alternative process for the preparation of the mixture of compounds of formula I said mixture comprising from 20 to 95% mol of compounds of formula I wherein both R groups are C₁₅ aliphatic groups is the simple mixture of the quaternary ammonium compounds of formula I in the defined proportions.

It is also possible to start from a mixture of the symmetrical ketones of formula VI R—C(═O)—R, wherein R groups are as defined above, in the defined proportions of C₁₅ and C₁₇ aliphatic groups, followed by a hydrogenation step (as described above in step b), then an esterification step (as described above in step c) and a condensation step (as described above in step d), optional step e, and step f.

With the same reasoning it is possible to start from a mixture of the secondary alcohols of formula V in the right proportions and then carry out the esterification (step c), condensation (d), optional step e, and step f.

Also, it is possible to start from a mixture of the monoesters of formula III as described above in the right proportions and then carry out the condensation (d), optional step e, and step f.

The exemplary processes described before are examples of suitable processes, i.e. there might be other suitable processes to synthesize the compounds in accordance with the present invention. The processes described hereinbefore are thus not limiting as far as the methods of manufacture of the compounds according to the present invention is concerned.

The mixture of compounds of formula I can be used as surfactants. Surfactants are compounds that lower the surface tension (or interfacial tension) between two non miscible liquids, a liquid and a gas or between a liquid and a solid. Surfactants may act as detergents, wetting agents, emulsifiers, foaming agents, and dispersants.

Surfactants are usually organic compounds that are amphiphilic, meaning they contain both hydrophobic groups (their tails) and hydrophilic groups (their heads). Therefore, a surfactant contains both a water-insoluble (or oil-soluble) component and a water-soluble component. Surfactants shall diffuse in water and adsorb at interfaces between air and water or at the interface between oil and water, in the case where water is mixed with oil. The water-insoluble hydrophobic group may extend out of the bulk water phase, into the air or into the oil phase, while the water-soluble head group remains in the water phase.

The adsorption of a cationic surfactant on negatively charged surfaces is an important property for such surfactants. This property is usually linked to the minimum concentration of surfactant needed to produce aggregation of a negatively charged cellulose nanocrystal (CNC, which is often used as reference material) suspension in aqueous media. Consecutive variation of size can be monitored and followed by dynamic light scattering (DLS).

Following the protocol described in E.K. Oikonomou et al., J. Phys. Chem. B, 2017, 121 (10), 2299-307 the adsorption properties of ammonium compounds can be investigated by monitoring the ratio X=[surfactant]/[CNC] or the mass fraction M=[surfactant]/([surfactant+[CNC]), at fixed [surfactant]+[CNC]=0.01 wt % in aqueous solution, required to induce the agglomeration of the cellulose nanocrystals.

The biodegradability of the compounds of the present invention can be determined in accordance with procedures described in the prior art and known to the skilled person. Details about one such method, OECD standard 301, are given in the experimental section hereinafter.

The mixture of compounds of formula I exhibits outstanding surfactant properties and biodegradability.

It can be used in various aqueous or hydro-alcoholic formulations as the sole ammonium compound exhibiting surfactant properties, i.e. no other mono-ammonium compound exhibiting surfactant properties and no di- or higher ammonium compound exhibiting surfactant properties are present in these formulations.

The Applicant has observed that, in aqueous or hydro-alcoholic formulations, the mixture of compounds of formula I structured generally in the form of lamellae, such as multilamellar vesicles. This lamellar structure resulted generally in aqueous or hydro-alcoholic formulations exhibiting a substantially higher viscosity than the same formulations but based on an ammonium surfactant which structures in the form of micelles. This higher viscosity is well adapted to some applications, while for some other applications a somewhat lower viscosity is desired.

All along this description as well as in the below working examples, any developed formula has to be understood as involving, if appropriate, all potential enantiomers and diastereoisomers. No specific stereochemistry is targeted, in the absence of specific mention, each presented chiral molecule is in the form of its racemic mixture.

Should the disclosure of any patents, patent applications, and publications which are incorporated herein by reference conflict with the description of the present application to the extent that it may render a term unclear, the present description shall take precedence.

### WORKING EXAMPLES

### Example (Comparative) 1) Synthesis of a Mixture Compounds of Formula I Having a 13% mol of Compounds of Formula I Wherein Both R Groups are C₁₅ Aliphatic Groups, From a C₁₆-C₁₈ Fatty Acids Mixture With C₁₆:C₁₈=33.7:65.3 wt % (or in Other Words Mixture of R—COOH With R═C₁₅:R═C₁₇=33.7:65.3 wt %).

All the reactions are conducted under an inert argon atmosphere.

### Step a. and b.: Piria Ketonization and Hydrogenation

The 2 first steps (Piria and hydrogenation) have been performed according to the protocol described in example 12 of the published patent application WO 2020/254337.

### Step c.: Secondary Alcohol Esterification With Chloroacetic Acid

In a three necked 500 mL round bottom flask equipped with a magnetic stirring devise, a heater, a temperature probe, a distillation apparatus connected to a receiver flask are added:
50 g (0.102 mole, 1 eq.) of C₃₁-C₃₅ mixture of secondary alcohols.
39.1 g of chloroacetic acid (0.41 mole, 4 eq.)

The reaction mixture is then heated to 120° C. and stirring is started (900 rpm stirring rate) once the reaction mixture has completely melted (around 105° C.).

The reaction mixture is then allowed to stir at 120° C. and reaction progress is followed up thanks to 1H NMR spectroscopy.

After 1h00 stirring at 120° C., NMR analysis shows a conversion level of 82%. In order to effectively remove water that is co-generated during the reaction and to displace the equilibrium toward esterification completion, a slight vacuum is applied to the reactor (800 mbar).

After additional 2h00 of stirring at 120° C. under 800 mbar, NMR analysis of the reaction crude shows a conversion level of 96%.

Reactor pressure is then decreased down to 30 mbar and the temperature of the reaction medium is further increased to 140° C. in order to distillate out the excess of chloroacetic acid.

Distillation at 140° C., 30 mbar is carried out until complete disappearance of chloroacetic acid as evidenced by 1H NMR analysis of the crude (<0.3 mol % of remaining chloroacetic acid in the crude).

At the end of the reaction, the pressure is re-established to 1 atm. and the reaction medium is allowed to cool down to room temperature.

56.7 g of product is recovered as a beige wax with the following composition: 98.9 wt % of mixture of chloroacetate esters, 1 wt % of starting mixture of fatty alcohols, 0.04 wt % of remaining chloroacetic acid.

The esterification yield taking into account purity is 97%.

The crude product is then engaged to the next quaternization stage.

¹H NMR (CDCl₃, 400 MHZ) δ (ppm): 4.93 (quint, J=6.0 Hz, 1H), 4.01 (s, 2H), 1.64-1.46 (m, 4H), 1.45-1.05 (m, 57 H (average number)), 0.86 (t, J=6.8 Hz, 6H).

¹³C NMR (CDCl₃, 101 MHZ) δ (ppm): 167.32, 77.21, 41.37, 34.17, 32.16, 29.93, 29.90, 29.87, 29.79, 29.72, 29.69, 29.60, 25.42, 22.92, 14.33.

### Step d.: Chloroacetate Ester Quaternization With Trimethylamine

In a 1 L double-jacketed reactor equipped with a mechanical stirrer (propeller with four inclined plows), a temperature probe, a condenser and which is connected to 2 consecutive traps containing respectively an aqueous solution of HCl (0.1 M) and activated charcoal are added:
56 g (0.099 mole, 1 eq.) of mixture of chloroacetate esters obtained from step c.
212 mL (180.6 g, 0.397 mole, 4 eq.) of trimethylamine/THF solution (13 wt %, ˜2 mol/L).

The reaction mixture is then allowed to stir at 40° C. (700 rpm stirring rate) and the reaction progress is followed thanks to 1H NMR spectroscopy.

After 2h00 stirring at 40° C., the conversion level of mixture of chloroacetate esters is around 66%.

After 4h00 stirring at 40° C., the conversion level increased to 85%.

In order to increase the reaction kinetic, the temperature of the reaction medium is increased further to 55° C. and after 2 additional hours of stirring at 55° C., the conversion level has reached 94%.

The reaction mass is then allowed to stir at 55° C. for additional 6h00 in order to complete the reaction.

At this stage the reaction crude composition is: 98 mol % of mixture of glycine betaine esters of formula I and 0.7 mol % of the starting mixture of chloroacetate esters.

The reaction medium is then allowed to cool down to room temperature and all the volatiles are removed under vacuum to afford 61.41 g of crude material as a beige wax with the following composition: 98.2 wt % of mixture of glycine betaine esters of formula 1, 0.9 wt % of mixture of fatty secondary alcohols and 0.8 wt % of mixture of chloroacetate esters corresponding to a yield of 97.4% taking into account the purity.

¹H NMR (CD₃OD, 400 MHZ) δ (ppm): 5.02 (quint, J=6.0 Hz, 1H), 4.41 (s, 2H), 3.35 (s, 9H), 1.68-1.52 (m, 4H), 1.50-1.05 (m, 57 H (average number)), 0.87 (t, J=7.2 Hz, 6H).

¹³C NMR (CD₃OD, 101 MHz) δ (ppm): 165.46, 78.81, 63.98, 54.43, 34.59, 32.83, 30.56, 30.53, 30.49, 30.44, 30.34, 30.24, 26.07, 23.53, 14.54.

### Example 2) Synthesis of a Mixture of Compounds of Formula I Having a 41% mol of Compounds of Formula I Wherein Both R Groups are C₁₅ Aliphatic Groups, From a C₁₆-C₁₈ Fatty Acids Mixture With C₁₆:C₁₈=60.9:38.2 wt % (or in Other Words Mixture of R—COOH With R═C₁₅:R═C₁₇=60.9:38.2 wt %).

All the reactions are conducted under an inert argon atmosphere.

### Steps a. and b.: Piria Ketonization and Hydrogenation

The 2 first steps (Piria and hydrogenation) have been performed according to the protocol described in example 13 of the published patent application WO 2020/254337.

### Step c.: Secondary Alcohol Esterification With Chloroacetic Acid

In a three necked 500 mL round bottom flask equipped with a magnetic stirring devise, a heater, a temperature probe, a distillation apparatus connected to a receiver flask are added:
82 g (0.173 mole, 1 eq.) of C₃₁-C₃₅ mixture of secondary alcohols.
66.2 g of chloroacetic acid (0.693 mole, 4 eq.)

The reaction mixture is then heated to 120° C. and stirring is started (1200 rpm stirring rate) once the reaction mixture has completely melted.

A slight vacuum (800 mbar) is applied in order to remove water that is co- produced by the reaction and to displace the equilibrium toward esterification completion.

The reaction mixture is allowed to stir at 120° C., 800 mbar during 3h40 and reaction progress is followed up thanks to 1H NMR spectroscopy.

After 3h00 reaction time, NMR analysis shows a conversion level of 96%.

The pressure is then decreased down to 10 mbar in order to distillate out chloroacetic acid excess and the distillation is carried out until complete disappearance of chloroacetic acid as evidenced by 1H NMR analysis of the crude (<0.3 mol % of remaining chloroacetic acid in the crude).

At the end of the distillation, the pressure is re-established to 1 atm. and the reaction medium is allowed to cool down to room temperature.

95 g of product is recovered as a beige wax with the following composition: 98.3 wt % of mixture of chloroacetate esters and 1.7 wt % of starting mixture of fatty alcohols.

The esterification yield taking into account purity is 98%.

The crude product is then engaged into the next quaternization stage.

¹H NMR (CDCl₃, 400 MHZ) δ (ppm): 4.93 (quint, J=5.6 Hz, 1H), 4.01 (s, 2H), 1.62-1.46 (m, 4H), 1.33-1.16 (m, 54.8 H (average number)), 0.86 (t, J=6.8 Hz, 6H).

### Step d.: Chloroacetate Ester Quaternization With Trimethylamine

In a 1L double-jacketed reactor equipped with a mechanical stirrer (propeller with four inclined plows), a temperature probe, a condenser and which is connected to 2 consecutive traps containing respectively an aqueous solution of HCl (0.1 M) and activated charcoal are added:
95 g (98.3 wt % purity, 0.17 mole, 1 eq.) of a mixture of chloroacetate esters obtained from step c.
364 mL (309 g, 0.68 mole, 4 eq.) of trimethylamine/THF solution (13 wt %, ˜2 mol/L).

The reaction mixture is then allowed to stir at 55° C. (1200 rpm stirring rate) and the reaction progress is followed thanks to 1H NMR spectroscopy.

After 3h30 stirring at 55° C., the conversion level of mixture of chloroacetate esters is around 87%.

After 5h45 stirring at 55° C., the conversion level increased to 97%.

The reaction mass is then allowed to stir at 55° C. for additional 6h00 in order to complete the reaction.

At this stage the reaction crude composition is: 98 mol % of mixture of glycine betaine esters of formula I and 0.2 mol % of the starting mixture of chloroacetate esters.

The reaction medium is then allowed to cool down to room temperature and all the volatiles are removed under vacuum to afford 103 g of crude material as a beige wax with the following composition: 98.3 wt % of mixture of glycine betaine esters of formula 1, 1.5 wt % of mixture of fatty secondary alcohols and 0.2 wt % of mixture of chloroacetate esters corresponding to a yield of 98%.

¹H NMR (CD₃OD, 400 MHZ) δ (ppm): 4.97 (quint, J=6.0 Hz, 1H), 4.38 (s, 2H), 3.36 (s, 9H), 1.65-1.46 (m, 4H), 1.45-1.05 (m, 54.8 H (average number)), 0.84 (t, J=6.8 Hz, 6H).

¹³C NMR (CD₃OD, 101 MHZ) δ (ppm): 164.81, 78.67, 63.64, 54.28, 34.12, 32.36, 30.11, 30.08, 30.05, 30.02, 29.90, 29.81, 29.78, 25.66, 23.10, 14.38.

### Biodegradability Assessment:

Readily biodegradability of the test substances have been measured according to the 301 F OECD protocol.

A measured volume of inoculated mineral medium, containing a known concentration of the test substance in order to reach about 50 to 100 mg ThOD/I (Theorical Oxygen Demand) as the nominal sole source of organic carbon, is stirred in a closed flask (oxitop™ respirometric flask) at a constant temperature (20±2° C.) for up to 28 days. Oxitop™ respirometric bottles were used in this test in order to access the biodegradability of the test samples: sealed culture BOD flasks were used at a temperature of 20±2° C. during 28 days.

Evolved carbon dioxide is absorbed by pellets of Natrium or Potassium hydroxide present in the head space of the bottle. The amount of oxygen taken up by the microbial population (=oxygen consumption expressed in mg/l) during biodegradation process (biological oxidation of the test substance) will decrease the pressure of the head space (ΔP measured by the pressure switch) and will mathematically be converted in mg O₂ consumed/litre. Inoculum corresponds to a municipal activated sludge washed in mineral medium (ZW media) in order to decrease the DOC (Dissolved Oxygen Carbon) content. Control solutions containing the reference substance sodium acetate and also toxicity control (test substance+reference substance) were used for validation purposes.

Reference substance, sodium acetate, has been tested in one bottle (at a nominal concentration of 129 mg/l corresponding to 100 mg ThOD/l in order to check the viability of the inoculum. Toxicity control corresponds to the mixture of the substance reference and the test substance; it will check if the test substance is toxic towards the inoculum (if so, the test has to be redone at a lower test substance concentration, if feasible regarding the sensitivity of the method).

As the test substances are for a majority of them not very soluble in water (if some are soluble in water, their metabolite after hydrolysis containing the alkyl chain is often very low soluble in water), we used a specific protocol named the “emulsion protocol”. This protocol enable us to increase the bioavailability of the poorly water soluble substance in the aqueous phase where we have the inoculum.

Emulsion protocol consists of adding the test substance in the bottle through a stock solution made in an emulsion.

Emulsion is a 50/50 v/v mixture of a stock solution of the test substance dissolved in a non biodegradable surfactant containing aqueous solution (Synperonic PE 105 at 1 g/l) and then mixed with a mineral silicone oil AR 20 (Sigma).

The first dissolution of the test substance in the non biodegradable surfactant containing aqueous solution often required magnetic stirrer agitation followed by ultrasonication.

Once the dissolution is made, we mix the aqueous solution with a mineral silicone oil at a 50/50 volume/volume ratio. This emulsion is maintained by magnetic stirrer agitation and is sampled for an addition in the corresponding bottle in order to reach the required test substance concentration.

Of course, 2 emulsion controls, are run in parallel during the test in order to remove their value from the emulsion bottle containing the test substance added through the emulsion stock solution.

The results of the biodegradability tests are summarized in the table 1 below:

TABLE 1

| Mixture of | mol % chain length | | | |
|---|---|---|---|---|
| Compounds of | distribution | | | Biodegradability rate vs DThO |
| formula I: | C₃₁ | C₃₃ | C₃₅ | after 28 days |
| Example 1 - | 13 | 46 | 40 | 50.4% (average over 2 |
| comparative | | | | replicates) |
| Example 2 - | 41 | 46 | 13 | 67.5% (average over 3 |
| invention | | | | replicates) |

As we can see on the table above, the mixture of quaternary ammonium compounds of formula I derived from the C₁₆-C₁₈ fatty acids mixture with C₁₆:C₁₈=60.9:38.2 wt % displays a final biodegradation rate of 67.5% and can be therefore considered as readily biodegradable. On the other hand the mixture of quaternary ammonium compounds of formula I derived from the C₁₆-C₁₈ fatty acids mixture with C₁₆:C₁₈=33.7:65.3 wt % displays a lower biodegradation rate of 50.4% and cannot be considered as readily biodegradable.

Those results show clearly that the hydrocarbon chain length distribution in the final mixture of compounds of formula I (and therefore in the starting mixture of fatty acids) has drastic impact on the biodegradability.

### Additional Experiments

Additional mixtures of quaternary ammonium compounds of formula I have been prepared by mixing the previous mixtures obtained at example 1 and 2 in two different ratios (one according to the invention and the other as comparative) and the biodegradability assessment has been performed according to the same method as described above. The results are indicated in Table 2 below.

TABLE 2

| | | | | Corresponding | | |
|---|---|---|---|---|---|---|
| Mix composition | | | | wt % fatty | | |
| (in weight % | | | | acid | | % Biodeg |
| of mixture of | mol % chain length | | | distribution | | vs DThO |
| Ex1: mixture | distribution | | | (in mol %) | | (average 2 |
| of Ex2) | C₃₁ | C₃₃ | C₃₅ | C₁₆ | C₁₈ | replicates) |
| EX 3) Comparative: | 18 | 46 | 36 | 39 | 61 | 57% |
| 83:17 | | | | (42) | (58) | |
| EX 4) Invention: | 23 | 46 | 31 | 43 | 57 | 61% |
| 67:33 | | | | (46) | (54) | |

### CONCLUSION

Readily biodegradation (meaning in this case BOD>60% vs. DThO after 28 days) for the mixture of quaternary ammonium compounds of formula I is obtained for a C₃₁ content in the hydrocarbon chain length distribution≥20 mol % (C₃₅ content≤31 mol %) corresponding to a C₁₆ fatty acid content in the starting fatty acid material≥45 mol % for a C₁₆-C₁₈ fatty acid mixture raw material.

In other words, readily biodegradation is obtained for an average hydrocarbon chain length≤C₃₃ in the mixture of quaternary ammonium compounds of formula I.

1. A mixture of compounds of formula I 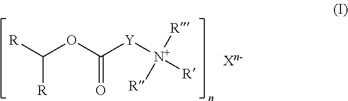
wherein R groups, which may be the same or different at each occurrence, are a C₁₅ or a C₁₇ aliphatic group,
Y is a divalent C₁-C₆ aliphatic group,
R′, R″ and R′″, which may be the same or different, are hydrogen or a C₁ to C₄ alkyl group,
Xⁿ⁻ is a counter-anion selected from the group consisting of
a halide and n is 1,
a hydrocarbylsulfate anion of formula R^{a}—O—SO₂—O⁻, wherein R^{a} denotes a C₁-C₂₀, is optionally halogenated, and n is 1,
a hydrocarbylsulfonate anion of formula R^{a}—SO₂—O⁻, wherein R^{a} denotes a C₁-C₂₀, is optionally halogenated, and n is 1,
a sulfate anion of formula SO₄²⁻ and n is 2,
a hydrogensulfate anion of formula HSO₄⁻, where n is 1,
a carbonate anion of formula CO₃²⁻ and n is 2,
a hydrogencarbonate anion of formula HCO₃⁻ and n is 1,
a dihydrogenphosphate anion of formula H₂PO₄⁻ and n is 1,
a hydrogenphosphate anion of formula HPO₄²⁻ and n is 2,
a phosphate anion of formula PO₄³⁻ and n is 3,
an organic carboxylate anion of formula R^{a}(CO₂⁻)ₙ wherein R^{a} denotes a C₁-C₂₀, is optionally substituted by an heteroatom containing group, and n is 1, 2, or 3,
and mixtures thereof,
n is an integer equal to 1, 2 or 3, depending on the nature of the counter-anion, and the mixture comprises from 20 to 95% mol of compounds of formula I wherein both R groups are C₁₅ aliphatic groups.

2. The mixture according to claim 1, wherein R groups are C₁₅ or C₁₇ alkyl groups and the mixture comprises from 20 to 95% mol of compounds of formula I wherein both R groups are C₁₅ alkyl groups.

3. The mixture according to claim 1, wherein R groups are C₁₅ or C₁₇ linear alkyl groups and the mixture comprises from 20 to 95% mol of compounds of formula I wherein both R groups are C₁₅ linear alkyl groups.

4. The mixture according to claim 1, wherein Y is a methylene group.

5. The mixture according to claim 1, wherein R′, R″ and R′″ are methyl.

6. The mixture according to claim 1, wherein Xⁿ⁻ is a halide and n is equal to 1.

7. The mixture according to claim 1, the mixture comprises from 20 to 60% mol of compounds of formula I wherein both R groups are C₁₅ aliphatic groups.

8. The mixture according to claim 1 comprising:
from 20 to 95% mol of compounds of formula I wherein both R groups are C₁₅ linear alkyl groups,
from 4.9 to 50% mol of compounds of formula I wherein one R group is a C₁₅ linear alkyl group and the other R group is a C₁₇ linear alkyl group, and
from 0.1 to 31% mol of compounds of formula I wherein both R groups are C₁₇ linear alkyl groups.

9. The mixture according to claim 1, further comprising less than 5% mol of compounds of formula I wherein at least one of the R groups is a C₇ to C₁₃ aliphatic group and/or C₁₉ to C₂₁ aliphatic group.

10. A process to produce a mixture of compounds of formula I as defined in claim 1,
wherein the process is starting from a mixture of fatty acids having a formula of R—COOH,
wherein R is a C₁₅ or C₁₇ aliphatic group, and
wherein the mixture of fatty acids comprises from 45 to 98% mol of fatty acids having the formula R—COOH, wherein R is a C₁₅ aliphatic group.

11. The process according to claim 10, wherein the process is starting from a mixture of fatty acids having the formula R—COOH, wherein R of the mixture of fatty acids is a C₁₅ or C₁₇ linear alkyl group, and wherein the mixture of fatty acids comprises from 45 to 78% mol fatty acids having the formula R—COOH, wherein R is a C₁₅ linear alkyl group.

12. A process according to claim 10, wherein the process comprises:
a. decarboxylative ketonization of a mixture of fatty acids R—COOH, wherein R is a C₁₅ or C₁₇ aliphatic group and wherein the mixture of fatty acids comprises from 45 to 98% mol of R—COOH with R being a C₁₅ aliphatic group, in the presence of a metal catalyst, thereby obtaining a mixture of internal ketones of formula VI: R—C(═O)—R (VI), wherein R groups of formula VI that are the same or different at each occurrence and are as defined above,
b. hydrogenating the mixture of internal ketones of formula VI obtained from step a. in the presence of hydrogen (H₂) and a catalyst, thereby obtaining a mixture of secondary alcohols of formula V: R—CH(OH)—R (V), wherein R groups of formula V are the same or different at each occurrence and are as defined above,
c. esterifying the mixture of secondary alcohols of formula V obtained at step b. with a carboxylic acid reagent of formula IV:
[L-Y—CO₂H]^{(t-1)-}[U^{u+}]_{(t-1)/u} (IV)
wherein L is a leaving group,
t is an integer which is equal to 1 or which is equal or superior to 2,
U^{u+} is a cation,
u is an integer fixing the positive charge of the cation,
Y is as defined in claim 1 or 4 and
R groups are as previously described,
thus obtaining a mixture of monoesters of formula III: 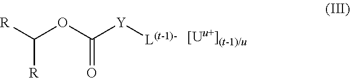
wherein R, Y, L, t, U, and u are as previously described,
d. condensing of the mixture of monoesters of formula III obtained at step c. with an amine of formula R′R″R′″N, wherein R′, R″ and R′″ which may be the same or different, are hydrogen or a C₁ to C₄ alkyl group to obtain a mixture of compounds of formula II: 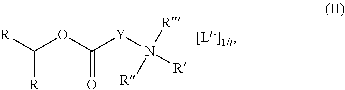
wherein R, R′, R″,R′″, Y, L and t are as previously described,
e. optionally exchanging an anion of the mixture of compounds of formula II by contacting the mixture of compounds of formula II obtained at step d. with a salt of formula [U′^{u′+}]_{n/u′}Xⁿ⁻ in order to substitute L^{t-} by Xⁿ⁻ when L^{t-} is different from Xⁿ⁻, X and n being as defined in claim 1 and U′^{u′+} is a cation, u′ is an integer fixing the positive charge of the cation, and
f recovering the mixture of compounds of formula I as defined in claim 1.

13. The process according to claim 12, wherein L^{t-} is equal to Xⁿ⁻ as defined in claim 1 and compounds of formula II are equal to compounds of formula I.

14. The process according to claim 12, comprising the step e. of anion exchange.

15. A surfactant composition comprising a mixture of compounds of formula I according to claim 1.

16. The process according to claim 10, wherein the mixture of fatty acids comprises from 55 to 71% mol of fatty acids having the formula R—COOH, wherein R is a C₁₅ linear alkyl group.

17. The mixture according to claim 1, wherein the mixture comprises from 30 to 50% mol of compounds of formula I having a C₁₅ aliphatic group for each R group.

18. The mixture according to claim 17, wherein the C₁₅ aliphatic group for each R group is an aliphatic alkyl group.

19. The mixture according to claim 1, wherein the mixture comprises:
from 20 to 60% mol of compounds of formula I having a C₁₅ linear alkyl group for each R group,
from 35 to 50% mol of compounds of formula I having one R group that is a C₁₅ linear alkyl group and the other R group is a C₁₇ linear alkyl group, and
from 5 to 31% mol of compounds of formula I having a C₁₇ linear alkyl group for each R group.

20. The mixture according to claim 1, wherein the mixture comprises:
from 30 to 50% mol of compounds of formula I having a C₁₅ linear alkyl group for each R group,
from 41 to 50% mol of compounds of formula I having one R group that is a C₁₅ linear alkyl group and the other R group is a C₁₇ linear alkyl group, and
from 9 to 20% mol of compounds of formula I having a C₁₇ linear alkyl group for each R group.
